(19) United States
(12) Patent Application Publication
(10) Pub. No.: US 2006/0074466 A1
(43) Pub. Date: Apr. 6, 2006

(54) Compositions having improved osteogenesis and methods for making and using same

(75) Inventor: Theodore I. Malinin, Key Biscayne, FL (US)

Correspondence Address: ROBERT W STROZIER, P.L.L.C, PO BOX 429, BELLAIRE, TX, 77402-0429, US

(73) Assignee: LOSTEC, INC.

(21) Appl. No.: 10/958,207

(22) Filed: Oct. 4, 2004

(51) Int. Cl. A61N 5/06

(52) U.S. Cl. 607/88

(57) ABSTRACT

An implantable bone-containing composition and a method for making same are disclosed. The compositions includes sterilized bone, which has been exposed to a dose of radiation in excess of about 45 kGy in the absence of radio-protective agents. The sterilized bone has higher osteogenic capacity than bone sterilized at the conventional dose of between 15 kGy and 35 kGy, approaching or surpassing the osteogenic capacity of the bone prior to irradiation.

### BACKGROUND OF THE INVENTION

1. Field of the Invention

The present invention relates to compositions including a relatively high dose radiation, sterilized bone-containing composition having usual or improved osteoinductive activity or osteogenic capability and methods for making and using same.

More particularly, the present invention relates to compositions having improved osteoinductive activity or osteogenic capability and methods for making and using same, where the compositions include a bone-containing material irradiated with a relatively high dose of ionizing radiation sufficient to improve an osteoinductive activity of the irradiated bone compared to an osteoinductive activity of bone irradiated with a conventional dose of ionizing radiation, where the relatively high dose of ionizing radiation is between about 45 kGy and about 100 kGy, and preferably, between about 50 kGy and about 75 kGy, and particularly, between about 50 kGy and about 70 kGy.

2. Description of the Related Art

It is well known in the art that exposure of bone to irradiation in doses sufficient for microbial deactivation leads to progressive, dose-dependant inactivation of the osteogenic potential of irradiated bone. (See Urist M R and Hernandez A., “Excitation transfer in bone; Deleterious effects of cobalt 60 radiation-sterilization of bank bone,” Arch. Surg. 109:586, 1974.) The doses of irradiation conventionally used to produce bactericidal-fungicidal effects vary from 15 kGy to 35 kGy. Further increases in the radiation dosage of the bone impair biomechanical integrity of musculoskeletal tissue allografts. (See Barbour S A, King W, “The safe and effective use of allograft tissue—an update.” Am. J. Sports Med. 31(5):791, 2003 and Triantafyllou N, Sotiropoulos E and Triantafyllou J N, “The mechanical properties of the lyophilized and irradiated bone grafts,” Acta Orth. Belgica 41 Suppl 1(1):35, 1974.) Therefore, doses of radiation in excess of 25 kGy are not recommended for sterilization of bone and tissue allografts. (See Zhang, Y, Homsi D, Gates K, Oakes K, Sutherland V, Wolfinbarger L, J R, “A comprehensive study of physical parameters, biomechanical properties and statistical correlation of iliac crest bone wedges used in spinal fusion surgery. IV. Effect of gamma irradiation on mechanical and material properties,” Spine 19(3):304, 1994.) Moreover, at these doses, 15 to 35 kGy, some viruses and allegedly some spore forming microorganisms may not be inactivated. Furthermore, retention of biomechanical integrity of bone and soft tissues (tendon, ligaments, fascias, etc.) has no relationship to the osteogenic potential of bone grafts.

Higher doses of radiation have been used to irradiate tissues treated, prior to irradiation with radioprotective agents. The radioprotective agents prevent biomechanical degradation of tissues resulting in reduction of biomechanical degradation changes observed in tissues irradiated with 25 kGy of radiation. (See King W D, Grieb T A, Fomg R Y, Lin J, Wolfinbarger L, Sharp C, and Drohan W, “Pathogen inactivation of soft tissue allografts using high dose gamma irradiation with early clinical results,” Proc. 2004 Ann. Mtg. AAOS 5:571, 2004.) Bone allografts treated with radio-protective agents and exposed to higher doses of radiation were reported to posses osteoinductive capacity comparable to that present in bone allografts irradiated with 25 kGy. (See Hollinger, J O, Rabinowitz N, Grieb T, Lin J, Fomg R Y and Burgess W, “Microbial sterility assurance in cadaveric bone by a process that preserves mechanical and biological integrity of allografts,” Clearant, Inc. Gaithersburg, Md., April 2004.) However, biological activity and toxicity of radioprotective agents such as dimethyl sulfoxide, propylene glycol, di-(2 quanidoethyl) disulfide dihydrobromide, methylalanine, etc. at the site of implantation are not well known and defined. Moreover, the effectiveness of the use of radioprotective compounds to protect bone in high dose irradiation applications is further limited because radioprotective agent are unable to penetrate dense bone, have toxicity problems and are not easily removed from the implantation site.

Thus, there is a need in the art for an improved radiation sterilization treatment that does not lower osteogenic activity and osteoinductive activity, but may indeed improve osteogenic activity and osteoinductive activity.

### SUMMARY OF THE INVENTION

The present invention avoids the limitations of the prior art because the method and resulting compositions are sterilized using a relatively high dose of ionizing radiation in the absence of a radio-protective agent, while improving the osteoinductive activity of bone relative to bone sterilized using a conventional lower dose of ionizing radiation. In fact, bone sterilized with relatively high doses of radiation show osteoinductive properties similar to unsterilized bone. The method includes the step of exposing a bone-containing material to an effective dose of ionizing radiation, where the effective dose is sufficient to sterilize the bone and to improve an osteoinductive activity of the irradiated bone compared to an osteoinductive activity of bone irradiated with a conventional dose of ionizing radiation, where the effective dose of ionizing radiation is between about 45 kGy and about 100 kGy, and preferably, between about 50 kGy and about 75 kGy, and particularly, between about 50 kGy and about 70 kGy and where the conventional dose of ionizing radiation is between 15 kGy and 35 kGy. The relatively high effective doses of ionizing radiation of this invention were found to insure complete microbial and viral inactivation, while surprisingly improving osteogenic capactiy and osteoinductive activity of particulate bone in the absence of radio-protective agents compared to bone irradiated at conventional doses of ionizing radiation, generally between 15 kGy and 35 kGy. The invention is equally applicable to autografts, allografts (homografts), xenografts or any other preparation including bone as one of its constituents for implantation or any mixture or combination thereof.

The present invention provides a composition including a bone-containing material having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower dose is between 15 kGy and 35 kGy.

The present invention also provides a composition including a bone material having been subjected to a relatively high dose of ionizing radiation sufficient to increase its osteoinductive activity compared to bone having been subjected to a conventional lower dose of ionizing radiation, where the relatively high dose is at least 45 kGy, preferably, at least 50 kGy and particularly, at least 54 kGy, with an upper limit below a radiation level known to completely alter the basic integrity and biochemical composition of bone. This upper limit is thought to be doses of radiation above about 100 kGy.

The present invention provides a bone implant including a bone-containing material having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower does is between 15 kGy and 35 kGy.

The present invention provides a bone graft including a bone-containing material having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower does is between 15 kGy and 35 kGy.

The present invention provides a non-demineralized (whole bone) and/or demineralized bone preparation and especially a particulate non-demineralized (whole bone) and/or demineralized bone preparation, where the preparation includes bone having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower does is between 15 kGy and 35 kGy. The biomechanical integrity of the preparations of this invention are not considered as important as is the preservation of osteoinductive activity of the preparation of this invention such as particulate bone grafts.

The present invention provides a non-demineralized (whole bone) and especially to particulate non-demineralized (whole bone) preparation, where the preparation includes bone having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower does is between 15 kGy and 35 kGy. The biomechanical integrity of the preparations of this invention are not considered as important compared to the preservation of osteoinductive activity of the composition of this invention such as particulate bone grafts.

The present invention provides a demineralized bone and especially to particulate demineralized bone preparation, where the preparation includes bone having been subjected to a relatively high dose of radiation sufficient to sterilize the bone and to increase its osteoinductive activity as compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and w here the conventional lower does is between 15 kGy and 35 kGy. The biomechanical integrity of the preparations of this invention are not considered as important compared to the preservation of osteoinductive activity of the composition of this invention such as particulate bone grafts.

Method of Preparation

The present invention provides a method including the step of irradiating a composition including a bone material with a relatively high dose of ionizing radiation sufficient to increase its osteoinductive activity compared to bone having been subjected to a conventional lower dose of ionizing radiation, where the sufficient dose is at least 45 kGy, preferably, at least 50 kGy and particularly, at least 54 kGy, with an upper limit below a radiation level known to damage the basic integrity of bone and where the conventional lower does is between 15 kGy and 35 kGy. This upper limit is thought to be doses of radiation above about 100 kGy.

The present invention provides a method including the step of irradiating a non-demineralized and/or demineralized bone composition with a relatively high dose of ionizing radiation sufficient to sterilize the bone and to increase its osteoinductive activity compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower dose is between 15 kGy and 35 kGy.

The present invention provides a method including the step of irradiating a non-demineralized and/or demineralized bone composition with a relatively high dose of ionizing radiation sufficient to sterilize the bone and to increase its osteoinductive activity compared to bone having been subjected to a conventional lower dose of ionizing radiation, where the relatively high dose is between about 54.2 kGy and about 65.6 kGy.

The present invention provides a method including the step of irradiating non-demineralized and/or demineralized bone with a relatively high dose of ionizing radiation sufficient to sterilize the bone and to increase its osteoinductive activity compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower dose is between 15 kGy and 35 kGy. The method also includes the step of implanting the irradiated bone into an animal including a human.

The present invention also provides a method including the step of implanting an irradiated bone composition of this invention into an animal including a human.

The present invention also provides a method for treating bone defects comprising the step of implanting a therapeutically effective amount of a composition of this invention into a bone defect of an animal including a human, where the composition has improved osteoinductive activity and where the composition induces healing of bone defect.

The present invention provides method of sterilization with relatively high doses of ionizing radiation of a micro-particulate cancellous and/or cortical non-demineralized and/or demineralized bone composition, where the particles range in size from about 355 microns to 25 microns and preferably, between about 125 microns to 25 microns. Micro-particulate bone compositions so sterilized are particularly suitable for making bone putty or bone jelly because such micro-particulate bond compositions are easily blended with water, saline or other substances and produce a composition similar in characteristic to toothpaste.

The present invention also provides a method including the steps of irradiating particulate bone preparations in a frozen state with a relatively high dose of ionizing radiation sufficient to sterilize the bone and to increase its osteoinductive activity compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower dose is between 15 kGy and 35 kGy. Following irradiation, these osteoinductive particulate bone preparations can be transplanted frozen directly into a bone defect site of an animal including a human or the preparations can be freeze-dried and stored for future implantation.

This invention also provides methods of making sterile particulate bone allografts, xenografts or autografts, where the bone have been sterilized with a relatively high dose of ionizing radiation sufficient to sterilize the bone and to increase its osteoinductive activity compared to bone having been subjected to a conventional lower dose of ionizing radiation, where the relatively high dose is between about 45 kGy and about 100 kGy, and preferably, between about 50 kGy and about 75 kGy, and particularly, between about 50 kGy and about 70 kGy, and where the conventional lower dose is between 15 kGy and 35 kGy.

### DESCRIPTION OF THE DRAWINGS

The invention can be better understood with reference to the following detailed description together with the appended illustrative drawings in which like elements are numbered the same:

FIG. 1 depict a photomicrograph of a defect filled with bone powder allograft sterilized with high dose radiation at six week post-implantation;

FIG. 2 depict a photomicrograph of a defect filled with bone powder allograft sterilized with conventional radiation at six week post-implantation;

FIG. 3A depicts an X-ray of an animal femur six weeks post implantation of irradiated particulate bone allograft;

FIG. 3B depicts an X-ray of an animal tibia having an implant sterilized with a high dose radiation evidencing healing at six week post transplantation and having an implant sterilized with a conventional dose of radiation evidencing no healing; and

FIG. 4 depict a photograph of bone defects filled with implant sterilized with a high dose of radiation where the defects evidenced by arrows have healed completely and are now filled with normal bone.

### DETAILED DESCRIPTION OF THE INVENTION

The inventors have found that bone and bone-containing materials can be irradiated with a relatively high dose of ionizing radiation in such a manner as to sterilize the bone or the bone in the bone-containing material and at the same time improve its osteoinductive activity compared to bone sterilized using a conventional lower dose of ionizing radiation. In fact, bone irradiated with relatively high doses of ionizing radiation has an osteoinductive activity similar to that of a nascent bone. The method works on all type of bone including whole bone, bone pieces, ground bone or any other form of bone, whether non-demineralized, demineralized or a mixture thereof. The method unexpectedly produces sterilized bone and bone-containing compositions with maintained or improved osteoinductive activity compared to bone treated with the conventional radiation dose. While the inventor has surprisingly found that relatively high doses of ionizing radiation are not detrimental to the osteoinductive activity of the treated bone, this finding is contrary to the known trend for irradiation of bone in the dose range between 15 kGy and 35 kGy, where a steady decrease in osteoinductive active is observed with increasing radiation doses. Currently, the inventor does not know precisely at what radiation dose osteoinductive activity bottoms out and begins to increase, but the inventor knows that at a doses between about 54.2 kGy and about 65.6 kGy, the osteoinductive activity of the irradiated bone is similar to the osteoinductive activity of the bone prior to irradiation. By similar the inventor means that the numeric value of the measured osteoinductive activity of the high dose irradiated bone is with about 10% of the osteoinductive activity of nascent bone. Thus, the inventor believes that in the radiation dose range between 35 kGy and 54.2 kGy, the reduction in osteoinductive activity bottoms out and then increases with increasing doses of radiation.

In particular, the inventor has found that particulate bone/bone powder/bone dust or micro-particulate bone graft compositions which include bone rendered sterile by exposure to a relatively high dose of ionizing radiation unexpectedly show much higher osteogenic capacity or osteoinductive activity compared to bone subjected to conventional radiation treatments. In fact, the osteoinductive capacity or osteogenic capacity of the particulate bone compositions exposed to relatively high doses irradiation between about 54.2 kGy and about 65.6 kGy is similar to an osteoinductive capacity or osteogenic capacity of non-irradiated bone. Moreover, these results were obtained in the absence of a radio-protective agent. The high doses of ionizing radiation renders the compositions of this invention such as autografts, allografts or xenografts sterile because these high doses of radiation inactivate microbial organisms including spore forming clostridia, fungi and viruses, while substantially maintaining osteoinductive activity (substantially meaning that the numeric values of osteoinductive activity are within about 10%). However, the effect of such high doses of radiation on prions is yet unknown.

The present invention broadly relates to the discovery that relatively high doses of ionizing radiation can be used to sterilize bone-containing compositions, especially, particulate bone-containing compositions. The relatively high doses are generally doses greater than or equal to about 45 kGy and preferably greater than or equal to about 50 kGy, but below a dose that would significantly damage the integrity of the bone such as dose above about 110 kGy. Preferred doses ranges are between about 45 kGy to about 100 kGy, preferably, between about 50 kGy and about 75 kGy, and particularly, between about 50 kGy and about 70 kGy of ionizing radiation Experimentally, a dose range of between about 54.2 kGy and about 65.6 kGy has been found to result in complete sterilization. Surprisingly, bone irradiated with such relatively high doses of ionizing radiation have improved osteoinductive activity compared to bone sterilized using a conventional radiation dose between about 15 kGy and about 35 kGy. In fact, relatively high dose bone treatments have been found to retain the bone's osteoinductive capacity to near nascent levels in the absence of radio-protective compounds. This invention is contrary to the commonly held belief that doses of radiation exceeding 25 kGy should not be used for sterilization of bone allografts, because in the dose range between about 15 kGy and 35 kGy, osteoinductive activity is known to steadily decrease. T he present invention allows for definitive sterilization of bone-containing compositions, especially particulate bone allografts, powdered bone, bone dust, gels containing bone dust, jellies containing bone dust and micro-particulate bone compositions, whether the bone is nondemineralized or demineralized without inhibiting osteogenesis. The compositions of this invention are well suited for use in spinal fusions, filling of cavities in bone, revisions of a hip, revisions of a knee and other arthroplasties, repairing cranio-facial defects, periodontal grafting, or other bone related operations and reconstructions. The method involves the exposure of bone or exposure of the bone-containing material to an effective dose of ionizing radiation sufficient to produce an osteoinductive activity in the irradiated bone substantially equal to an osteoinductive activity of the bone prior to irradiation, preferably, the exposure is at least 45 kGy, and particularly, at least 50 kGy of ionizing radiation. The preferred ionizing radiation dose is generally between about 45 kGy to about 100 kGy, preferably, between about 50 kGy and about 75 kGy, and particularly, between about 50 kGy and about 70 kGy of ionizing radiation.

The present invention broadly relates to a composition including a bone-containing material, where the bone has been subjected to a relatively high dose of radiation sufficient to sterilize the bone and to maintain or increase the bone's osteoinductive activity as compared to radiation treatments of bone using a dose of radiation between 15 kGy and 35 kGy. Preferably, the relatively high dose of radiation is at least 45 kGy 50 kGy. Particularly, the relatively high dose of radiation is at least 50 kGy of ionizing radiation. The preferred ionizing radiation dose is generally between about 45 kGy to about 100 kGy, preferably, between about 50 kGy and about 75 kGy, and particularly, between about 50 kGy and about 70 kGy of ionizing radiation, with an experimentally verified range between about 54.2 kGy and 65.6 kGy.

The present invention provides a method including the step of irradiating a bone component of a bone-containing material or the bone-containing material with a relatively high dose of radiation sufficient to maintain or increase an osteoinductive activity of the bone within the bone-containing material compared to bone radiation treatments using a dose of radiation between 15 kGy and 35 kGy. Preferably, the relatively high dose of radiation is at least 45 kGy 50 kGy. Particularly, the relatively high dose of radiation is at least 50 kGy of ionizing radiation. The preferred ionizing radiation dose is generally between about 45 kGy to about 100 kGy, preferably, between about 50 kGy and about 75 kGy, and particularly, between about 50 kGy and about 70 kGy of ionizing radiation, with an experimentally verified range between about 54.2 kGy and 65.6 kGy.

The present invention provides a method including the step of introducing a composition of this invention into a bone defect, site of bone reconstruction or other site requiring a bone-containing composition capable of osteogenesis, where the composition induces osteogenesis at the site.

Suitable bone material for use in this invention include, without limitation, whole bone, whole bone fragments, ground whole bone, demineralized bone, demineralized bone fragments, ground demineralized bone, or mixtures or combinations thereof. Bone material also includes bio-compatible matrices including, without limitation, whole bone, whole bone fragments, ground whole bone, demineralized bone, demineralized bone fragments, ground demineralized bone, or mixtures or combinations thereof. Bone material also includes auto grafts, allografts (homografts) and xenografts including, without limitation, whole bone, whole bone fragments, ground whole bone, demineralized bone, demineralized bone fragments, ground demineralized bone, or mixtures or combinations thereof.

Suitable irradiation procedures include, without limitation, any type of ionizing radiations with sufficient penetrating depth to sterilize bone such as cobalt 60 irradiation, strontium 90 irradiation, gamma ray irradiation, hard X-ray irradiation, beta particle irradiation, alpha particle irradiation, positron irradiation, proton and anti-proton irradiation, or the like, or mixture or combination of any of these irradiation procedures.

### Experimental Section

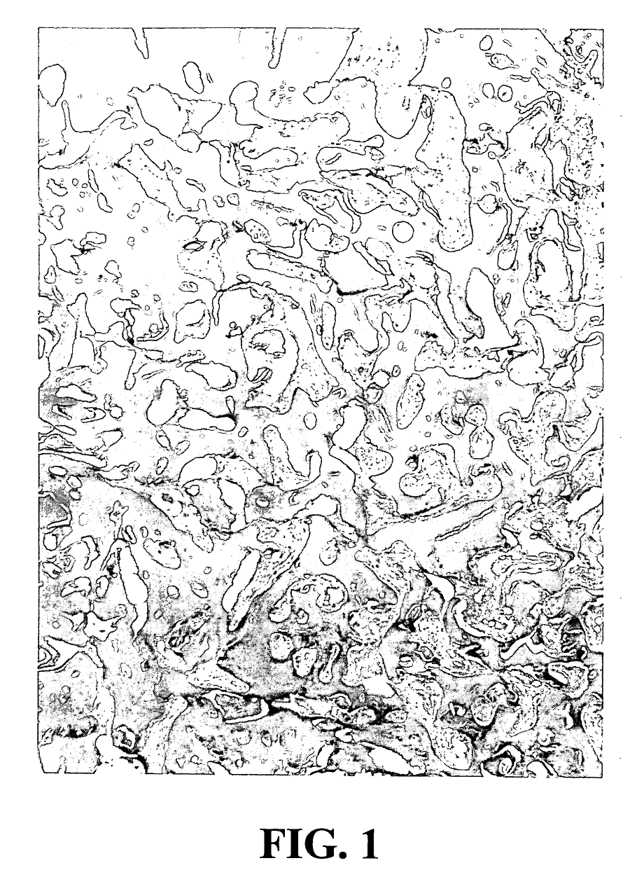

Unexpectedly, the inventor has found that bone particles (bone powder) allografts irradiated at radiation doses between about 54.2 kGy and 65.6 kGy using ionizing radiation induced new bone formation, observed osteogenic activity, when implanted in artificially created defects in long bones of non-human primates. The allografts have not been treated with any radio-protective chemical agents or substances prior or subsequent to irradiation. After implantation of the composition into a bone defect, the new bone was formed around each bone particle of the composition, and within six weeks of implantation the entire defect was obliterated as shown in FIG. 1.

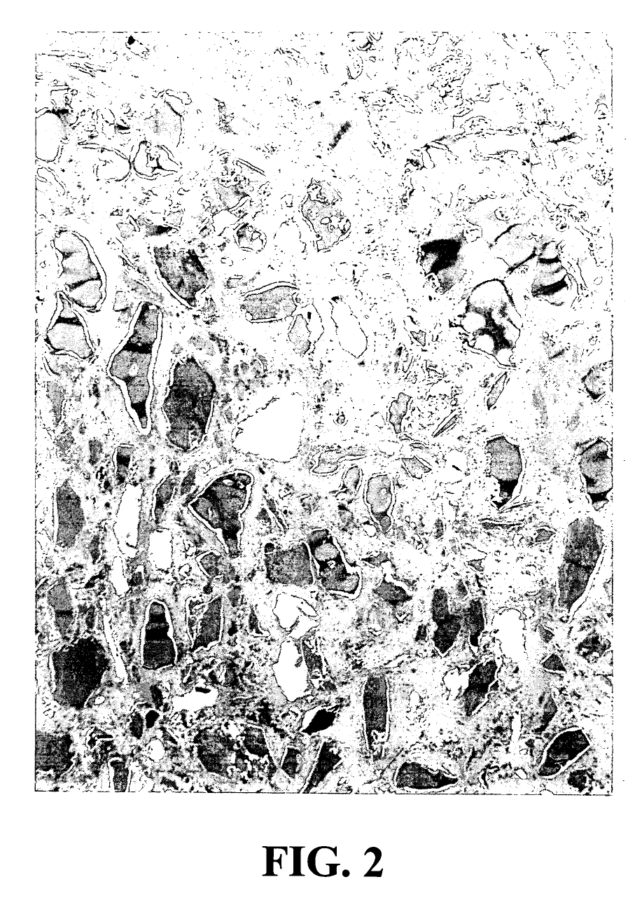

This finding is in sharp contrast to the healing that usually occurs with transplanted particulate bone irradiated using the conventional ionizing radiation dose of 25 kGy. As shown FIG. 2, six weeks after implantation of such a conventionally sterilized particular bone composition, many of the particles remain unaltered, with healing of the defect progressing only slowly from peripheries of the defect.

Thus, a bone-containing material exposed to relatively high doses of ionizing radiation clearly out performs an analogous b one-containing material exposed to conventional doses of ionizing radiation. This finding is somewhat paradoxical. One of ordinary skill in the art would expect progressive diminution of the osteoinductive capacity of bone-containing material such as grafts with increasing doses of irradiation, as happens in radiation dose ranges between 15 kGy and 35 kGy. Instead, bone-containing material such as bone-containing material including particulate bone irradiated with radiation doses exceeding 50 kGy showed more osteoinductive activity than did its counterparts irradiated with 25 kGy.

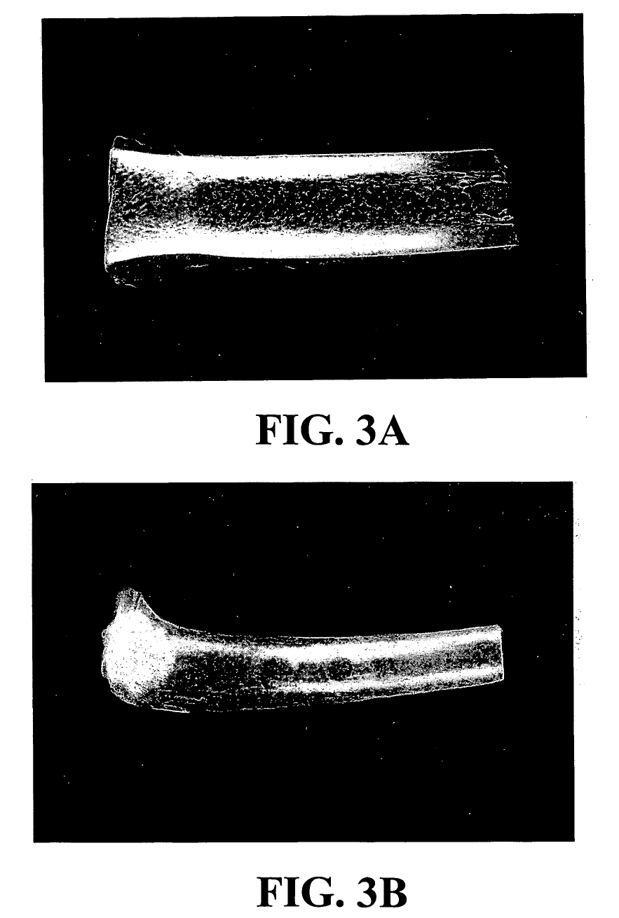

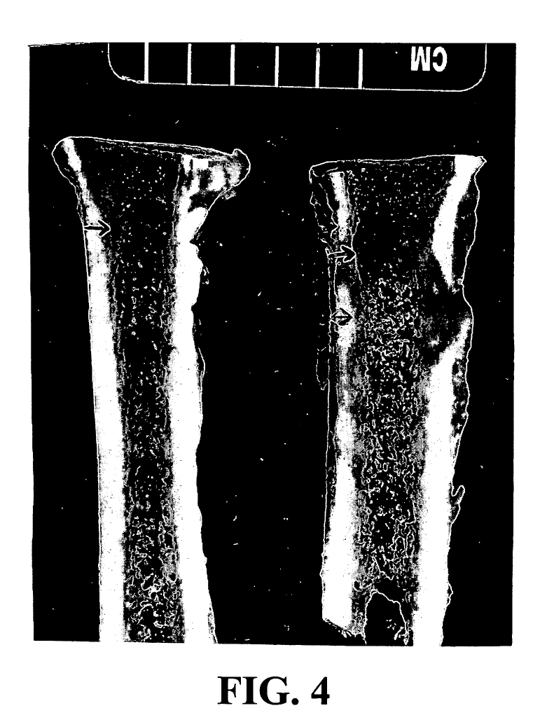

Referring now to FIG. 3, radiologically, bone powder allografts of this invention treated with relatively high doses of radiation show healing of bone defects in which they were placed. Likewise gross specimens of bone show healing and replacement of the defect with new bone as shown in FIG. 4.

Allografts are harvested from experimental animals and are ground to a desired size in a bone mill. The ground allografts are washed in saline, and are then rapidly frozen in liquid nitrogen vapor at a temperature between about −150° C. to −120° C. Before irradiation, the allografts are transferred to containers containing solid carbon dioxide at a temperature of about −76° C. The bone is then irradiated while frozen in dry ice. After irradiation, the bone is either stored or freeze-dried. The irradiated particulate bone can then be directly implanted or mixed with a bio-compatible carrier and implanted. The freeze-dried material can also be directly implanted or mixed with a bio-compatible carrier and implanted.

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that may be made which do not depart from the scope and spirit of the invention as described above and claimed hereafter.

I claim:

1. A method comprising the step of:
irradiating a composition including non-demineralized bone, demineralized bone or mixtures or combinations thereof with a relatively high dose of ionizing radiation in the absence of radio-protective agents,
where the relatively high dose sterilizes the bone and increases an osteoinductive activity of the bone compared to an osteoinductive activity of bone irradiated with a conventional lower dose of ionizing radiation.

2. The method of claim 1, wherein the relatively high dose of ionizing radiation is at least 45 kGy and the conventional lower dose of ionizing radiation is between 15 kGy and 35 kGy.

3. The method of claim 1, wherein the relatively high dose of ionizing radiation is at least 50 kGy of ionizing radiation and the conventional lower dose of ionizing radiation is between 15 kGy and 35 kGy.

4. The method of claim 1, wherein the relatively high dose of ionizing radiation is at least 54 kGy of ionizing radiation and the conventional lower dose of ionizing radiation is between 15 kGy and 35 kGy.

5. The method of claim 1, wherein the relatively high dose of ionizing radiation is between about 45 kGy to about 100 kGy and the conventional lower dose of ionizing radiation is between 15 kGy and 35 kGy.

6. The method of claim 1, wherein the relatively high dose of ionizing radiation is between about 50 kGy and about 75 kGy and the conventional lower dose of ionizing radiation is between 15 kGy and 35 kGy.

7. The method of claim 1, wherein the relatively high dose of ionizing radiation is between about 50 kGy and about 70 kGy and the conventional lower dose of ionizing radiation is between 15 kGy and 35 kGy.

8. The method of claim 1, wherein the relatively high dose of ionizing radiation is between about 54.2 kGy and about 65.6 kGy and the conventional lower dose of ionizing radiation is between 15 kGy and 35 kGy.

9. The method of claim 1, wherein the relatively high dose insures complete microbial and viral inactivation.

10. The method of claim 1, wherein the composition is selected from the group consisting of autografts, allografts, xenografts, other bone-containing compositions and mixtures or combinations thereof.

11. A method for sterilizing bone comprising the step of:
exposing a bone-containing composition including non-demineralized bone, demineralized bone or mixtures or combinations thereof to an effective dose of ionizing radiation in the absence of radio-protective agents,
where the effective dose at least 45 kGy and is sufficient to sterile the composition and to improve an osteoinductive activity in the irradiated bone compared to bone irradiated with a conventional lower dose of ionizing radiation between 15 kGy and 35 kGy.

12. The method of claim 11, wherein the osteoinductive activity of the irradiated bone is substantially similar to an osteoinductive activity of non-irradiated bone.

13. The method of claim 11, wherein the effective dose is at least 50 kGy.

14. The method of claim 11, wherein the effective dose is at least 54 kGy.

15. The method of claim 1 1, wherein the effective dose is between about 45 kGy to about 100 kGy.

16. The method of claim 11, wherein the effective dose is between about 50 kGy and about 75 kGy.

17. The method of claim 11, wherein the effective dose is between about 50 kGy and about 70 kGy.

18. The method of claim 11, wherein the effective dose is between about 54.2 kGy and about 65.6 kGy.

19. The method of claim 11, wherein the effective dose insures complete microbial and viral inactivation.

20. The method of claim 11, wherein the composition is selected from the group consisting of autografts, allografts, xenografts, other bone-containing compositions and mixtures or combinations thereof.

21. A method comprising the steps of:
irradiating a composition including non-demineralized bone, demineralized bone or mixtures or combinations thereof with a relatively high dose of ionizing radiation in the absence of radio-protective agents, and
implanting the composition into a bone site in an animal including a human,
where the relatively high dose sterilizes the bone and increases an osteoinductive activity of the bone compared to an osteoinductive activity of bone irradiated with a conventional lower dose of ionizing radiation.

22. The method of claim 21, wherein the relatively high dose of ionizing radiation is at least 45 kGy and the conventional lower dose of ionizing radiation is between 15 kGy and 35 kGy.

23. The method of claim 21, wherein the relatively high dose of ionizing radiation is at least 50 kGy of ionizing radiation and the conventional lower dose of ionizing radiation is between 15 kGy and 35 kGy.

24. The method of claim 21, wherein the relatively high dose of ionizing radiation is at least 54 kGy of ionizing radiation and the conventional lower dose of ionizing radiation is between 15 kGy and 35 kGy.

25. The method of claim 21, wherein the relatively high dose of ionizing radiation is between about 45 kGy to about 100 kGy and the conventional lower dose of ionizing radiation is between 15 kGy and 35 kGy.

26. The method of claim 21, wherein the relatively high dose of ionizing radiation is between about 50 kGy and about 75 kGy and the conventional lower dose of ionizing radiation is between 15 kGy and 35 kGy.

27. The method of claim 21, wherein the relatively high dose of ionizing radiation is between about 50 kGy and about 70 kGy and the conventional lower dose of ionizing radiation is between 15 kGy and 35 kGy.

28. The method of claim 21, wherein the relatively high dose of ionizing radiation is between about 54.2 kGy and about 65.6 kGy and the conventional lower dose of ionizing radiation is between 15 kGy and 35 kGy.

29. The method of claim 21, wherein the relatively high dose insures complete microbial and viral inactivation.

30. The method of claim 21, wherein the composition is selected from the group consisting of autografts, allografts, xenografts, other bone-containing compositions and mixtures or combinations thereof.

31. A composition comprising non-demineralized bone, demineralized bone or mixtures or combinations thereof, where the bone has been irradiated with a relatively high dose of ionizing radiation sufficient to sterilize the bone and to increase an osteoinductive activity of the bone as compared to bone having been irradiated with a conventional lower dose of ionizing radiation.

32. The composition of claim 31, the relatively high dose of ionizing radiation is at least 45 kGy and the conventional lower dose of ionizing radiation is between 15 kGy and 35 kGy.

33. The composition of claim 31, wherein the relatively high dose of ionizing radiation is at least 50 kGy of ionizing radiation and the conventional lower dose of ionizing radiation is between 15 kGy and 35 kGy.

34. The composition of claim 31, wherein the relatively high dose of ionizing radiation is at least 54 kGy of ionizing radiation and the conventional lower dose of ionizing radiation is between 15 kGy and 35 kGy.

35. The composition of claim 31, wherein the relatively high dose of ionizing radiation is between about 45 kGy to about 100 kGy and the conventional lower dose of ionizing radiation is between 15 kGy and 35 kGy.

36. The composition of claim 31, wherein the relatively high dose of ionizing radiation is between about 50 kGy and about 75 kGy and the conventional lower dose of ionizing radiation is between 15 kGy and 35 kGy.

37. The composition of claim 31, wherein the relatively high dose of ionizing radiation is between about 50 kGy and about 70 kGy and the conventional lower dose of ionizing radiation is between 15 kGy and 35 kGy.

38. The composition of claim 31, wherein the relatively high dose of ionizing radiation is between about 54.2 kGy and about 65.6 kGy and the conventional lower dose of ionizing radiation is between 15 kGy and 35 kGy.

39. The composition of claim 31, wherein the relatively high dose insures complete microbial and viral inactivation.

40. The composition of claim 31, wherein the composition is selected from the group consisting of autografts, allografts, xenografts, other bone-containing compositions and mixtures or combinations thereof.

41. A composition comprising a bone-containing material including non-demineralized bone, demineralized bone or mixtures or combinations thereof, where the bone has been irradiated with a relatively high dose of ionizing radiation sufficient to sterilize the bone and to increase an osteoinductive activity of the bone as compared to bone having been irradiated with a conventional lower dose of ionizing radiation.

42. The composition of claim 41, the relatively high dose of ionizing radiation is at least 45 kGy and the conventional lower dose of ionizing radiation is between 15 kGy and 35 kGy.

43. The composition of claim 41, wherein the relatively high dose of ionizing radiation is at least 50 kGy of ionizing radiation and the conventional lower dose of ionizing radiation is between 15 kGy and 35 kGy.

44. The composition of claim 41, wherein the relatively high dose of ionizing radiation is at least 54 kGy of ionizing radiation and the conventional lower dose of ionizing radiation is between 15 kGy and 35 kGy.

45. The composition of claim 41, wherein the relatively high dose of ionizing radiation is between about 45 kGy to about 100 kGy and the conventional lower dose of ionizing radiation is between 15 kGy and 35 kGy.

46. The composition of claim 41, wherein the relatively high dose of ionizing radiation is between about 50 kGy and about 75 kGy and the conventional lower dose of ionizing radiation is between 15 kGy and 35 kGy.

47. The composition of claim 41, wherein the relatively high dose of ionizing radiation is between about 50 kGy and about 70 kGy and the conventional lower dose of ionizing radiation is between 15 kGy and 35 kGy.

48. The composition of claim 41, wherein the relatively high dose of ionizing radiation is between about 54.2 kGy and about 65.6 kGy and the conventional lower dose of ionizing radiation is between 15 kGy and 35 kGy.

49. The composition of claim 41, wherein the relatively high dose insures complete microbial and viral inactivation.

50. The composition of claim 41, wherein the composition is selected from the group consisting of autografts, allografts, xenografts, other bone-containing compositions and mixtures or combinations thereof.

51. The method of claim 1, wherein the bone is particulate bone including particles have a particle size less than or equal to 355μ.

52. The method of claim 11, wherein the bone is particulate bone including particles have a particle size less than or equal to 355μ.

53. The method of claim 21, wherein the bone is particulate bone including particles have a particle size less than or equal to 355μ.

54. The composition of claim 31, wherein the bone is particulate bone including particles have a particle size less than or equal to 355μ.

55. The composition of claim 41, wherein the bone is particulate bone including particles have a particle size less than or equal to 355μ.
